(19) United States
(12) Patent Application Publication
(10) Pub. No.: US 2010/0055179 A1
(43) Pub. Date: Mar. 4, 2010

(54) Composition of and Method for Preparing Orally Disintegrating Tablets Containing a High Dose of Pharmaceutically Active Ingredients

(75) Inventors: Jae Han Park, Olivette, MO (US); Stephen H. Wu, Chesterfield, MO (US); Kevin M. Holman, Lebanon, IL (US); Glenn A. Bish, St. Louis, MO (US); Donald G. Krieger, Collinsville, IL (US); Daniel S. Ramlose, Red Bud, IL (US)

Correspondence Address: Mallinckrodt Inc., 675 McDonnell Boulevard, HAZELWOOD, MO, 63042, US

(73) Assignee: MALLINCKRODT INC., Hazelwood, MO (US)

(21) Appl. No.: 12/519,927

(22) PCT Filed: Dec. 20, 2007
(86) PCT No.: PCT/US2007/026185

§ 371 Date: Jun. 18, 2009

Related U.S. Application Data
(60) Provisional application No. 60/876,383, filed on Dec. 21, 2006.
(60) Provisional application No. 60/948,208, filed on Jul. 6, 2007.
(60) Provisional application No. 60/952,638, filed on Jul. 30, 2007.

(51) Int. Cl. A61K 9/20

(52) U.S. Cl. 424/465

(57) ABSTRACT

The present invention is directed to improved compositions and methods for preparing orally disintegrating tablets (ODTs). In one aspect of the present invention, the ODT further contains at least one active pharmaceutical ingredient (API). In another aspect of the present invention, the ODT contains a high load of at least one API. Specifically, the ODTs described in this invention containing a high load of API can accommodate up to about 70% w/w of active pharmaceutical ingredient in a unit dosage, while exhibiting the desirable attributes of fast disintegration time, acceptable hardness and friability for push through blister and bottle packages, and acceptable mouth feel.

### FIELD OF THE INVENTION

The present invention generally relates to compositions of and methods for the preparation of orally disintegrating tablets (ODTs). More specifically, the invention is directed to compositions of and methods for the preparation of ODTs containing a high dose of a pharmaceutically active ingredient (API).

### BACKGROUND OF THE INVENTION

Tablets and capsules are unit dosage forms commonly used for oral administration. It has been reported that more than 35% of the US population has experienced some level of difficulty in swallowing these conventional dosage forms, particularly pediatric and geriatric patients. There is a need for a solid dosage form that rapidly dissolves or disintegrates in the mouth. The dosage form is generally called orally disintegrating tablets (ODTs), which can be taken without chewing or the need for water. Because of the ease of administration and patient compliance, ODTs are especially beneficial for pediatric and geriatric patients and patients with dysphagia.

A number of commercial products employing specific manufacturing technologies are available. For example, Zydis® from Cardinal Health is prepared by a freeze drying method; FlashDose® from Biovail is prepared by “cotton candy spinning” and compression; AdvaTab® from Eurand is prepared by direct compression of non-effervescent excipients with a combination of an external lubrication system; and OraSolv® or DuraSolv® from Cima is prepared by direct compression of effervescent excipients. These commercial ODT products using various technologies, however, show several disadvantages including requirement of specialized packaging due to weak tablet strength, complex and expensive manufacturing process, and slow disintegration time for tablets made by a conventional direct compression method. Furthermore, the amount of API in current commercial ODT products is typically less than 50 mg in the unit dosage form.

More specifically, the process of freeze drying requires water to be removed by sublimation from the product preparation step. This method creates an amorphous porous structure that can be rapidly dissolved in the mouth. The drug loading of an ODT made using a freeze drying method is typically limited to less than 50 mg. In addition, the mechanical strength of the tablets made thereof is usually very poor so that the tablets require specialized blister packages.

Molding is another method for making ODTs. The process requires heat and solvents, including water. The molded ODT can provide a fast disintegration time in the mouth because of the porous matrix and water soluble materials embedded in the matrix. Molded ODTs, however, typically have poor mechanical strength, require a complicated manufacturing process, and have high production costs. ODTs prepared by a direct compression method of effervescent materials are highly sensitive to moisture and therefore require specialized packaging to protect the tablet from moisture. Additionally, effervescent ODTs typically exhibit an unpleasant mouth feel and a slower disintegration time.

Another method for producing ODTs is to employ a direct compression method under low compression force. The direct compressible ODTs typically contain combinations of sugars, superdisintegrants, starch, cellulose derivatives, and inorganic salts. The disintegration time is typically greater than 40 seconds at a certain mechanical strength, which does not meet with the FDA disintegration specification of less than 30 seconds according to the USP method. The tablets produced by this process exhibit a high degree of friability, chalky taste and dry mouth feel when placed in the mouth. Another noticeable disadvantage is the poor mechanical strength of the tablets. Further, ODTs produced by direct compression lose the desirable characteristics of hardness, friability, disintegration time, and mouth feel when APIs like acetaminophen are applied to it.

Thus a need remains for an improved ODT formulation and method of manufacture. Specifically, there is need for improved formulations and methods of manufacture for ODTs containing a high dose of API in a finished unit dosage form.

### SUMMARY OF THE INVENTION

An illustrative aspect of the present invention is to provide an improved orally dissolving tablet. The improved ODT comprises at least one water-insoluble hydrophobic inorganic salt in combination with at least one water-insoluble inorganic salt, and at least one active pharmaceutical ingredient.

In another illustrative aspect of the present invention, there is provided an ODT composition comprising about 18% to about 88% by weight (w/w) of at least one water-soluble excipient; about 1% to about 20% w/w of at least one water swellable polymeric material; about 3% to about 25% w/w of at least one water-insoluble hydrophobic inorganic salt; about 1% to about 25% w/w of at least one water-insoluble inorganic salt; and at least one active pharmaceutical ingredient. The particle size of the water swellable polymeric material(s) and the water-insoluble inorganic salt(s) and the water-insoluble hydrophobic inorganic salt(s) is typically not more than about 80 pm by Malvern particle size analysis.

In yet another aspect of the present invention there is provided a method of making orally disintegrating granules. The method comprises granulating a mixture that includes at least one water-soluble excipient, at least one water swellable polymeric material, at least one water-insoluble hydrophobic inorganic salt, and at least one water-insoluble inorganic salt with water to form wet granules. The wet granules are dried to form substantially dry granules, and the substantially dry granules are screened (or milled) to produce orally disintegrating granules of a desired size.

In yet a further aspect of the present invention there is provided a method of making orally disintegrating granules. The method comprises granulating a mixture including about 18% to about 90% w/w of at least one water-soluble excipient; about 1% to about 20% w/w of at least one water swellable polymeric material; about 3% to about 25% w/w of at least one water-insoluble hydrophobic inorganic salt; and about 1% to about 25% w/w of at least one water-insoluble inorganic salt with either water or a polymeric binder solution to form wet granules. The wet granules are then substantially dried and screened (or milled) to a desired size.

In still another aspect of the present invention there is provided a method of making a rapidly disintegrating tablet. The method comprises granulating a mixture including about 18% to about 88% w/w of at least one water-soluble excipient, about 1% to about 20% w/w of at least one water swellable polymeric material, about 3% to about 25% w/w of at least one water-insoluble hydrophobic inorganic salt, and about 1% to about 25% w/w of at least one water-insoluble inorganic salt with water to form wet granules. The wet granules are then substantially dried and screened (or milled) to a desired size. The granules are then compressed into a tablet.

### DETAILED DESCRIPTION OF THE INVENTION

The present invention is directed to improved compositions and methods for preparing orally disintegrating tablets (ODTs). In one aspect of the present invention, the ODT further contains at least one active pharmaceutical ingredient (API). In another aspect of the present invention, the ODT contains a high load of at least one API. Specifically, the ODTs described in this invention containing a high load of API can accommodate up to about 70% w/w of APIs in a unit ODT dosage form, while exhibiting the desirable attributes of fast disintegration time, acceptable hardness and friability for push through blister and bottle packages, and acceptable mouth feel.

In one embodiment, the invention relates to the composition of orally disintegrating granules consisting of four components: (1) water soluble excipients, (2) water insoluble/swellable polymeric excipients, (3) water insoluble hydrophobic inorganic salts, and (4) water insoluble inorganic salts, that are less hydrophobic than component 3. Particularly the granules described in the invention consist of about 18% to about 90% w/w of Component 1; about 1% to about 20% w/w of Component 2; about 3% to about 30% w/w of Component 3; and about 1% to about 30% w/w of Component 4. Additionally, the ratio of water insoluble hydrophobic inorganic salts to the water insoluble inorganic salts typically ranges from about 1:10 to about 10:1. Suitable water soluble excipients are non-hygroscopic, or have a low degree of hygroscopicity. Disintegrants suitable for use in the present invention include but are not limited to sodium starch glycolate, croscarmellose sodium, crospovidone, low substituted hydroxypropyl cellulose, polyplasdone XL-10 (ISP technologies Inc.), and mixtures thereof. Suitable water insoluble hydrophobic inorganic salts include but are not limited to calcium diphosphate (dihydrate) with a particle size less than about 40 μm, calcium triphosphate, Talc Imperial USP BC (Mineral and Pigment Solution Inc., MPSI) with a particle size smaller than about 50 μm and a mean particle size of about 3 μm, and Talc Lo-Micron USP BC (MPSI) with a particle size smaller than about 50 μm and a mean particle size of about 1.2 μm. Suitable water insoluble hydrophobic inorganic salts include but are not limited to calcium silicate (Zeopharm 600 from HUBER Engineered Materials) with a particle size less than about 20 μm, hydrophobically modified calcium silicate (RxCipients FM1,000 from HUBER Engineered Materials) with a particle size smaller than about 50 μm, and Talc USP BC 300 (from MPSI) with a particle size smaller than about 80 μm and a mean particle size of about 15 μm. In another embodiment, the ODT may optionally include an additive. Suitable additives include but are not limited to colorants, sweeteners, flavorants, binders, lubricants and mixtures thereof.

In accordance with this invention, the orally disintegrating granules can be made by a conventional wet granulation process of either a mixture of the said components 1, 2, 3, and 4 or a mixture of the components 1, 2, 3, 4 and an API(s). The method for preparing the granule is comprised of dry blending of a powder mixture with or without APIs, wet granulation by spraying 15-60% w/w of water or a polymeric binder solution in a wet granulator, and drying the wet mass in a dryer, or optionally milling prior to dry screening with a #20 mesh sieve. Typically, the polymeric binder solution comprises fully pregelatinized starch and water soluble polymers. The amount of polymeric binder by weight generally ranges from about 0.1% to about 25% w/w. In one embodiment, for example, the amount of polymeric binder is between about 0.5% and about 15% w/w. Generally, the mean particle size of the screened dry granule is less than about 300 μm. In one embodiment, the mean particle size of the dry granule is between about 20 μm and about 300pm. In another embodiment, the mean particle size of the dry granule is between about 40 μm and about 275 μm. In yet another embodiment, the mean particle size of the dry granule is between about 50 μm and about 225 μm. In still another embodiment, the mean particle size of the dry granule is between about 60 μm and about 175 μm. In a further embodiment, the mean particle size of the dry granule is between about 70 μm and about 150 μm. In another embodiment, the mean particle size of the dry granule is between about 80 μm and about 120 μm. In still another embodiment, the mean particle size of the dry granule is between about 90 μm and about 110 μm. The orally disintegrating granules of the present invention exhibit rapid disintegration on tableting and other functionalities suitable for making ODT products.

Another aspect of the invention relates to preparing ODT products containing a high load of APIs. The ODTs can be prepared by direct compression of a dry blend of API(s) (some APIs can be granulated with the four components of the orally disintegrating granules), water insoluble inorganic salts with a particle size less than about 50 μm, a superdisintegrant with particle size less than about 70 μm, relatively hydrophilic lubricants such as sodium stearyl fumarate and magnesium stearate monohydrate, the granule described above, and any optional additives. In order for ODTs with a high API load to have a relatively short disintegration time of less than 30 seconds, water insoluble inorganic salts with a particle size less than about 50 μm and a superdisintegrant with particle size less than about 70 μm are necessary in the final blend when more than 30% w/w of API(s) are included. In one embodiment, the orally disintegrating granules typically have a bimodal particle size distribution consisting of a first mode representing a particle size group of 1 μm-80 μm and a second mode representing a particle size group of 70 μm-700 μm.

When less than 30% w/w API(s) are added to the blend, the water insoluble inorganic salts and superdisintegrant are optional in the final blend. The preferred particle size range of coated APIs for taste masking is between about 100 μm and about 250 μm for consistent compression during tableting and fast disintegration time. Depending on API characteristics and how the API is treated, the ODT of the present invention can accommodate up to about 70% w/w of either uncoated API(s) or coated API(s) for taste masking without losing desirable ODT attributes. The ODTs described herein provide a disintegration time of less than 30 seconds according to the USP disintegration test (FDA disintegration time specification for ODTs), less than 45 seconds in the Wetting test, which is an in vitro test, simulating disintegration time in the mouth, and have a low friability of less than about 0.5% (0.8% by weight-FDA friability specification for ODTs) at a tablet hardness of greater than about 5 kP so that the ODT tablets are suitable for packaging in conventional HDPE bottles and push through blister packages. The ODTs of the present invention can be made by using any conventional manufacturing equipment such as blenders, wet granulators, dryers, mills, and tablet presses.

API's useful in the present invention include but are not limited to the group consisting of non-steroidal anti-inflammatory agents, contraceptives, opioids, thyroid and antithyroid drugs, gout therapy drugs, cough and cold drugs, anticonvulsants, antirheumatic drugs, anti-migraine drugs, anti-parasite, hormonal drugs, mitotic inhibitors, immunosuppressants, antihypersensitive agents, calcium-channel blocking agents, antidepressants, anxiolytics, neurodegenerative disease drugs, bismuth salts, coagulants, antiulcer agents, coronary vasodilators, peripheral vasodilators, oral antibacterial and antifungal agents, antispasmodics, antitussive agents, antiasthmatic agents, bronchodilators, diuretics, muscle relaxants, brain metabolism altering drugs, tranquilizers, beta blockers, antiarrhythmic agents, anticoagulants, antiepileptic agents, antiemetics, hypo- and hypertensive agents, sympathomimetic agents, expectorants, oral antidiabetic agents, circulatory agents, nutritional supplements, pollakiuria remedies, angiotension-converting enzyme inhibitors, antiviral agents, antihistamines, and nasal decongestants.

The amount of API contained in the unit dosage form can vary. Typically, the amount of API contained in the ODTs of the present invention ranges from about 0.05% to about 70.0% w/w. In one embodiment, the amount of API contained in the ODT ranges from about 0.1% to about 65% w/w. In another embodiment, the amount of API contained in the ODT ranges from about 0.5% to about 55% w/w. In yet another embodiment, the amount of API contained in the ODT ranges from about 0.1 % to about 50% w/w. In still another embodiment, the amount of API contained in the ODT ranges from about 0.15% to about 45% w/w. In a further embodiment, the amount of API contained in the ODT ranges from about 0.2% to about 40% w/w.

Acetaminophen (APAP), for example, is commonly used as an analgesic. APAP is also used in combination with other pain medicines such as hydrocodone and oxycodone in the final dosage form. Typical levels of APAP are 650mg for adults and 325mg for children in a unit dosage form. When APAP is formulated with either oxycodone or hydrocodone, each unit dose is typically 400mg APAP. It is extremely difficult to include such a high level of API(s) in an ODT dosage form, while retaining most, if not all, desirable characteristics of mechanical strength, fast disintegration time both in the USP specified test and in the mouth, and acceptable mouth feel. In general, a blend having a high level of coated taste masked APAP or other API(s) typically causes the loss of the required ODT characteristics. In accordance with this invention, four different ODTs containing a high level of APAP were prepared and evaluated as shown in the examples below. The four different types of APAP are: (1) regular, non-coated APAP containing >99% acetaminophen with a particle size between about 10 μm and about 75 μm, (2) regular, non-coated APAP granulated with the four key components of the invention in a high shear wet granulator and subsequently dried on a tray, (3) COMPAP 273 (Mallinckrodt Inc.) spray dried compressible APAP containing 94.5% paracetamol, 0.5% crospovidone, and 5% pregelatinized starch with a mean particle size of about 100 μm, and (4) coated, taste masked APAP (Schwarz Pharma) coated with 20% ethylcellulose containing 80% paracetamol, with a mean particle size of about 165 μm.

### Definitions

The term “water insoluble hydrophobic inorganic salts” as used herein refers to an inorganic solid in powder form that absorbs water not more than 0.2% (w/w) at a relative humidity of 95% at 25° C. and has a particle size of about 40 μm or less.

The term “water insoluble inorganic salts” as used herein refers to an inorganic solid in powder form that absorbs water between 0.3% and 3.0% at relative humidity of 95% at 25° C., and has a particle size less than 50 μm.

The term “water insoluble/swellable excipients” as used herein refers to a disintegrant that absorbs water and swells rapidly in contact with water.

The term “water soluble excipients” as used herein refers to a solid material or a mixture of readily water soluble materials such as sugar, spray-dried sugar alcohols including mannitol, xylitol, and erythritol, and mixtures thereof.

### Examples

### Example 1

This example illustrates the preparation of fast orally disintegrating granules. The compositions of the respective formulations are shown in Table 1.

TABLE 1
Compositions of fast disintegrating granules

| | % (w/w) | | | | |
|---|---|---|---|---|---|
| | Formu- | Formu- | Formu- | Formu- | Formu- |
| Ingredient | lation A | lation B | lation C | lation D | lation E |
| Spray dried mannitol | 64.0 | 75.0 | 72.5 | 67.5 | 64.0 |
| (Pearlitol 200SD) | | | | | |
| Crospovidone XL-10 | 13.0 | 13.0 | 13.0 | 13.0 | 13.0 |
| Talc Imperial | 7.0 | 7.0 | 7.0 | 7.0 | 7.0 |
| (hydrophobic) | | | | | |
| Talc USP BC 300 | 16.0 | — | — | — | — |
| (less hydrophobic) | | | | | |
| Calcium silicate | — | 5.0 | 7.5 | 12.5 | 16.0 |
| (Zeopharm, less | | | | | |
| hydrophobic) | | | | | |

The granules were made by the following procedures: (1) 10 Kg of the ingredients based on the compositions of the respective formulations were dry blended in a 16 quart-V-blender (Twin shell) for 20 minutes, (2) 900 g of the dry blended Formulation A or 700 g of the dry blended Formulations B, C, D, and E was granulated in the 5L mixing bowl of a Glatt high shear wet granulator under specified conditions such as 20%-55% (w/w) of water atomized onto the powder bed over 5-20 minutes at impeller speeds of 50-200 rpm, chopper speed of 1,500 rpm and main blade speeds of 50-200 rpm, chopper speed of 1,500 rpm during granulation phase after water spray for 2-5 minutes, (3) the obtained wet mass was then passed through a No. 4 sieve to break the lumps, (4) the screened wet mass was air dried on an aluminum tray in a hood for three days, (5) after drying, the granulation was screened through a No. 20 sieve. These granules were blended with different APAP types mentioned previously, and compressed to form acetaminophen-containing ODTs. The particle size and size distribution of fast disintegrating granules were determined by using a Malvern particle size analyzer. The particle size distribution of the placebo granules of Formulation A was 2% by volume (w/v) of 9 μm-23 μm, 89% of 23 μm-316 μm, and 9% of greater than 316 μm with a mean particle size of 137 μm. The particle size distribution of the placebo granules of Formulation B was 4% (w/v) of 5 μm-30 μm, 96% (w/v) of 30 μm-416 μm with a mean particle size of 115 m. The particle size distribution of the placebo granules of Formulation C was 4% (w/v) of 4 μm-26 μm, 96% (w/v) of 26 μm-363 μm with a mean particle size of 112 μm. The particle size distribution of the placebo granules of Formulation D was 2% (w/v) of 2 μm-15 μm, 25% (w/v) of 17 μm-52 μm, and 73% (w/v) of 52 μm -360 μm with a mean particle size of 100 μm. The particle size distribution of the placebo granules of Formulation E was 4% (w/v) of 2 μm-11 μm, 28% (w/v) of 11 μm-52 μm, 64% (w/v) of 52 μm-416 μm, and 4% (w/v) of greater than 550 μm with a mean particle size of 99 μm.

### Example 2

This example illustrates the preparation of fast disintegrating tablets using regular APAP.

1 Kg of APAP blends were prepared by mixing the granules of the Formulation A in Example 1 with 0.3-15.0% (w/w) of regular APAP in a 4quart-V-blender for 20 minutes. The APAP blends were then lubricated with 1.0% (w/w) of sodium stearyl fumarate (PRUV, JRS Pharma) for 5 minutes. The lubricated blends were compressed into tablets using a pre-compression force of 1,000 newtons (N), and main compression forces of 3 kilonewtons (kN)-18 kN at 60 rpm in a 0.4062 inch die with flat faced and beveled edge punches on a 16 station Manesty Betapress. The target weight of each tablet was 400 mg. The physical properties of the tablets were evaluated according to these measurement procedures:

(1) Hardness test-The ODT tablet crushing load, which is the force (kilopond, kP) required to break a tablet into halves by compression in the diametral direction, was measured with a hardness tester (Varian Hardness Tester, VK-200).

(2) Friability test-The friability test method was performed by a Varian Friabilator according to the USP tablet friability method described in <1216> Tablet Friability of the General chapters describing General Test Assays.

(3) In vitro disintegration test-The disintegration time of the tablets was determined according to U.S. Pharmacopeia, Test No. 701.

(4) Wetting Test-One Whatman filter disc (21 mm in diameter) was placed in each well of a Corning 12-well polystyrene microplate (22 mm in diameter). One and a half millimeters of Sensient Blue #1 dye solution (similar to in vivo conditions-tongue surface) was then added into each well. An ODT tablet was carefully placed on the surface of the wet paper disc in each well using a pair of forceps, tablet face flat on the filter paper. Finally, the total wetting time was recorded as the time required for the blue dye solution to cover the surface of the tablet as simulated in vivo disintegration time. The physical characteristics of the tablets are shown in Table 2.

TABLE 2

| | Hardness | Friability | USP-DIT | Wetting time |
|---|---|---|---|---|
| % (w/w) APAP | (kP) | (%) | (sec.)* | (sec.) |
| 0 | 8.8 | 0 | 16 | 14 |
| 0.3 | 8.1 | 0.02 | 18 | 12 |
| 1.0 | 9.3 | 0.03 | 20 | 11 |
| 10.0 | 6.5 | 0.46 | 9 | 10 |
| 15.0 | 6.8 | 0.08 | 10 | 9 |

*In vitro disintegration time according to the USP method.

When the amount of APAP was greater than 15% (w/w), the tablets could not be prepared due to poor powder flow in the tablet press. Therefore, the ODT preparation using granule Formulation A can accommodate regular APAP up to 15% (w/w) or about 60 mg in a 400 mg tablet.

### Example 3

This example illustrates the preparation of fast disintegrating tablets using granulated regular APAP.

A powder mixture of Formulation A was dry blended with 0.3-30.0% (w/w) of regular APAP in a 4 quart-V-blender for 20 minutes. 900 g of the APAP blend was granulated in the 5L mixing bowl of a Glatt high shear wet granulator under specified conditions, such as 20% (w/w)-35% (w/w) of water over 10 minutes to 20 minutes at an impeller speed of 200 rpm and a chopper speed of 1,500 rpm, and an impeller speed of 200 rpm with a chopper speed of 1,500 rpm after water spray for 2 minutes. The obtained wet mass was then passed through a No. 4 sieve to break the lumps and the screened wet mass was then air dried on an aluminum tray in a hood for three days. The dried granules containing APAP were screened through a No. 20 sieve. 1 Kg of the APAP granules was lubricated with 1.0% (w/w) of sodium stearyl fumarate in a 4 quart-V-blender for 5 minutes. The lubricated blends were compressed into tablets and the physical properties of the tablets were evaluated according to the procedures described in Example 2. The physical characteristics of the tablets are shown in Table 3.

TABLE 3

| | Hardness | Friability | USP-DIT | Wetting time |
|---|---|---|---|---|
| % (w/w) APAP | (kP) | (%) | (sec.)* | (sec.) |
| 0.3 | 8.1 | 0.3 | 12 | 18 |
| 1.0 | 8.6 | 0.2 | 12 | 21 |
| 10.0 | 8.5 | 0.0 | 18 | 30 |
| 20.0 | 7.5 | 0.2 | 19 | 25 |
| 30.0 | 7.9 | 0.0 | 28 | 21 |

*In vitro disintegration time according to the USP method.

When the level of APAP was greater than 30% (w/w), the tablets could not be prepared because of high ejection forces during the tableting process. Therefore, the ODT preparation according to Example 3 can accommodate regular APAP as a granulated form up to 30% (w/w) or about 120 mg in a 400 mg tablet.

### Example 4

This example illustrates the preparation of fast disintegrating tablets using compressible COMPAP as the source of APAP.

1 Kg of COMPAP blends were prepared by mixing the granules of Formulation A with 0.3-50.0% of (w/w) APAP as COMPAP in a 4 quart-V-blender for 20 minutes. The COMPAP blends were then lubricated with 1.0% (w/w) of sodium stearyl fumarate (PRUV, JRS Pharma) for 5 minutes. The lubricated blends were compressed into tablets and the physical properties of the tablets were evaluated according to the procedures described in Example 2. The physical characteristics of the tablets are shown in Table 4.

TABLE 4

| | Hardness | Friability | USP-DIT | Wetting time |
|---|---|---|---|---|
| % (w/w) APAP | (kP) | (%) | (sec.)* | (sec.) |
| 0.3 | 7.9 | 0.5 | 26 | 15 |
| 1.0 | 7.6 | 0.3 | 15 | 16 |
| 10.0 | 8.0 | 0.5 | 11 | 22 |
| 20.0 | 9.1 | 0.0 | 11 | 30 |
| 30.0 | 7.2 | 0.5 | 16 | 45 |
| 40.0 | 5.7 | 0.0 | 16 | 65 |
| 50.0 | 4.2 | 0.3 | 16 | 117 |

*In vitro disintegration time according to the USP method.

The results showed that the ODT preparation using granule Formulation A can accommodate COMPAP up to 50% (w/w) or about 200 mg in a 400 mg tablet.

### Example 5

This example illustrate the preparation of fast disintegrating tablets using taste masked coated APAP (TM-APAP) from Schwarz Pharma.

1 Kg of TM-APAP blends were prepared by mixing the granules of Formulation A with 0.375-62.5% of (w/w) TM-APAP (equivalent to 0.3-50.0% APAP) in a 4 quart-V-blender for 20 minutes. The TM-APAP blends were then lubricated with 1.0% (w/w) of sodium stearyl fumarate (PRUV, JRS Pharma) for 5 minutes. The lubricated blends were compressed into tablets and the physical properties of the tablets were evaluated according to the procedures described in Example 2. The physical characteristics of the tablets are shown in Table 5.

TABLE 5

| % APAP | | | | |
|---|---|---|---|---|
| (% TM-APAP) | Hardness | Friability | USP-DIT | Wetting time |
| (w/w) | (kP) | (%) | (sec.)* | (sec.) |
| 0.0 (0.0) | 8.1 | 0.3 | 13 | 16 |
| 0.3 (0.375) | 4.9 | 0.0 | 11 | 17 |
| 1.0 (1.25) | 6.1 | 0.3 | 11 | 18 |
| 10.0 (12.5) | 5.5 | 0.0 | 9 | 14 |
| 20.0 (25.0) | 5.6 | 0.2 | 15 | 13 |
| 30.0 (37.5) | 6.1 | 0.3 | 14 | 16 |
| 40.0 (50.0) | 6.1 | 0.0 | 18 | 25 |
| 50.0 (62.5) | 6.3 | 0.0 | 60 | 50 |
| 50.0 (62.5)** | 5.9 | 0.3 | 34 | 44 |

*In vitro disintegration time according to the USP method.
**50% TM-APAP blend contained additional 5% of crospovidone XL-10 in the final blend.

These results showed that the ODT preparation using granule Formulation A could barely accommodate taste-masked APAP up to 50% (w/w) or about 160 mg in a 400 mg tablet.

### Example 6

This example illustrates the preparation of fast disintegrating tablets using TM-APAP from Schwarz Pharma and granule Formulation E.

1 Kg of TM-APAP blends were prepared by mixing the granules of Formulation E with 0.375-62.5% (w/w) of TM-APAP (equivalent to 0.3-50.0% APAP) in a 4 quart-V-blender for 20 minutes. The TM-APAP blends were then lubricated with 1.0% (w/w) of sodium stearyl fumarate (PRUV, JRS Pharma) for 5 minutes. The lubricated blends were compressed into tablets and the physical properties of the tablets were evaluated according to the procedures described in Example 2. The physical characteristics of the tablets are shown in Table 6.

TABLE 6

| % (w/w) APAP | Hardness | Friability | USP-DIT | Wetting time |
|---|---|---|---|---|
| (% TM-APAP) | (kP) | (%) | (sec.)* | (sec.) |
| 0.0 (0.0) | 5.1 | 0.3 | 8 | 14 |
| 0.3 (0.375) | 5.1 | 0.2 | 8 | 16 |
| 1.0 (1.25) | 5.4 | 0.2 | 8 | 17 |
| 10.0 (12.5) | 5.8 | 0.0 | 9 | 16 |
| 20.0 (25.0) | 5.3 | 0.3 | 11 | 12 |
| 30.0 (37.5) | 5.7 | 0.3 | 9 | 12 |
| 40.0 (50.0) | 5.5 | 0.2 | 13 | 16 |
| 50.0 (62.5) | 5.8 | 0.0 | 29 | 25 |
| 50.0 (62.5)** | 5.5 | 0.2 | 18 | 22 |

*In vitro disintegration time in the USP method.
**50% TM-APAP blend contained additional 5% of crospovidone XL-10 in the final blend

The results showed that the ODT preparation using granule Formulation E can accommodate taste-masked APAP up to 62.5% (w/w) or about 200 mg APAP in a 400 mg tablet.

### Example 7

This example illustrates the preparation of fast disintegrating tablets of TM-APAP from Schwarz Pharma and granule Formulations B, C, D and E.

1 Kg of TM-APAP blends were prepared by mixing the granules of Formulations B, C, D, E respectively with 62.5% of (w/w) TM-APAP (equivalent to 50% pure APAP) in a 4 quart-V-blender for 20 minutes. The TM-APAP blends were then lubricated with 1.0% (w/w) of sodium stearyl fumarate (PRUV, JRS Pharma) for 5 minutes. The lubricated blends were compressed into on a 16 station Manesty Betapress at a pre-compression force of 1,000 newtons (N) and main compression forces of 3 kilonewtons (kN)-18 kN at 60 rpm in a 0.5625 inch die with round, flat faced and beveled edge punches. The target weight of each tablet was 900 mg. The physical properties of the tablets were evaluated according to the procedures described in Example 2. The physical characteristics of the tablets are shown in Table 7.

TABLE 7

| | Hardness | Friability | USP-DIT | Wetting time |
|---|---|---|---|---|
| | (kP) | (%) | (sec.) * | (sec.) |
| Formulation 1 ^{a} | 8.9 | 0.27 | 45 | 27 |
| Formulation 2 ^{b} | 8.6 | 0.37 | 65 | 28 |
| Formulation 3 ^{c} | 8.8 | 0.31 | 79 | 28 |
| Formulation 4 ^{d} | 8.8 | 0.40 | 40 | 26 |

* In vitro disintegration time according to the USP method.
^{a} Formulation 1- tablets from a mixture of 62.5% of Schwarz's TM-APAP as received, 36.5% of the granules of Formulation B, and 1.0% of sodium stearyl fumarate.
^{b} Formulation 2- tablets from a mixture of 62.5% of Schwarz's TM-APAP as received, 36.5% of the granules of Formulation C, and 1.0% of sodium stearyl fumarate.
^{c} Formulation 3- tablets from a mixture of 62.5% of Schwarz's TM-APAP as received, 36.5% of the granules of Formulation D, and 1.0% of sodium stearyl fumarate.
^{d} Formulation 4- tablets from a mixture of 62.5% of Schwarz's TM-APAP as is, 36.5% of the granules of Formulation E, and 1.0% of sodium stearyl fumarate.

The results showed that the ODT preparation using granule Formulation B, C, D, and E can accommodate taste-masked APAP up to 62.5% (w/w) or about 450 mg in a 900 mg tablet. However, because of the tablet size, the respective disintegration times were longer than 30 seconds according to the USP method, but the respective wetting times were still shorter than 30 seconds.

### Example 8

This example illustrate the preparation of fast disintegrating tablets of using Schwarz TM-APAP in a particular size range and granule Formulation B, C, D, and E.

1 Kg of TM-APAP blends were prepared by mixing the granules of the formulations B, C, D, E respectively with 62.5% (w/w) of screened TM-APAP (equivalent to 50% pure APAP) in a 4 quart-V-blender for 20 minutes. The particle size range of the screened TM-APAP was between 150 μm and 250 μm on sieve analysis (pass through a No. 60 sieve and retained on a No. 100 sieve). The screened TM-APAP blends were then lubricated with 1.0% (w/w) of sodium stearyl fumarate (PRUV, JRS Pharma) for 5 minutes. The lubricated blends were compressed into 900 mg tablets and the physical properties of the tablets were evaluated according to the procedures described in Example 2. The physical characteristics of the tablets are shown in Table 8.

TABLE 8

| | Hardness | Friability | USP-DIT | Wetting time |
|---|---|---|---|---|
| | (kP) | (%) | (sec.) * | (sec.) |
| Formulation 1 ^{a} | 8.6 | 0.48 | 47 | 25 |
| Formulation 2 ^{b} | 8.6 | 0.47 | 44 | 24 |
| Formulation 3 ^{c} | 8.9 | 0.56 | 37 | 25 |
| Formulation 4 ^{d} | 8.5 | 0.3 | 26 | 23 |

* In vitro disintegration time in the USP method.
^{a} Formulation 1- tablets from a mixture of 62.5% of screened Schwarz's TM-APAP, 36.5% of the granules of Formulation B, and 1.0% of sodium stearyl fumarate.
^{b} Formulation 2- tablets from a mixture of 62.5% of screened Schwarz's TM-APAP, 36.5% of the granules of Formulation C, and 1.0% of sodium stearyl fumarate.
^{c} Formulation 3- tablets from a mixture of 62.5% of screened Schwarz's TM-APAP, 36.5% of the granules of Formulation D, and 1.0% of sodium stearyl fumarate.
^{d} Formulation 4- tablets from a mixture of 62.5% of screened Schwarz's TM-APAP, 36.5% of the granules of Formulation E, and 1.0% of sodium stearyl fumarate.

These results indicated that the respective disintegration times, comparing with the data in Example 7, were improved for TM-APAP with the size in the specified range.

### Example 9

This example illustrates the preparation of fast disintegrating tablets using Schwarz TM-APAP in a particular size range and an additional amount of disintegrant with specified particle size.

1 Kg of TM-APAP blends were prepared by mixing the granules of the formulations B, C, D, E with 62.5% (w/w) screened TM-APAP (equivalent to 50% pure APAP) and 5% of Crospovidone XL10 in a 4 quart-V-blender for 20 minutes. The particle size of the screened TM-APAP was between 150 μm and 250 μm on sieve analysis (passed through a No. 60 sieve and retained on a No. 100 sieve). The blends were then lubricated with 1.0% (w/w) of sodium stearyl fumarate (PRUV, JRS Pharma) for 5 minutes. The lubricated blends were compressed into tablets and the physical properties of the tablets were evaluated according to the procedures described in Example 2. The physical characteristics of the tablets were shown in Table 9.

TABLE 9

| | Hardness | Friability | USP-DIT | Wetting time |
|---|---|---|---|---|
| | (kP) | (%) | (sec.) * | (sec.) |
| Formulation 1 ^{a} | 7.7 | 1.0 | 27 | 30 |
| Formulation 2 ^{b} | 8.1 | 0.4 | 27 | 28 |
| Formulation 3 ^{c} | 9.3 | 0.3 | 22 | 26 |
| Formulation 4 ^{d} | 8.6 | 0.2 | 22 | 23 |

* In vitro disintegration time in the USP method.
^{a} Formulation 1- tablets from a mixture of 62.5% of screened Schwarz's TM-APAP, 31.5% of the granule of Formulation B, and 1.0% of sodium stearyl fumarate.
^{b} Formulation 2- tablets from a mixture of 62.5% of screened Schwarz's TM-APAP, 31.5% of the granule of Formulation C, and 1.0% of sodium stearyl fumarate.
^{c} Formulation 3- tablets from a mixture of 62.5% of screened Schwarz's TM-APAP, 31.5% of the granule of Formulation D, and 1.0% of sodium stearyl fumarate.
^{d} Formulation 4- tablets from a mixture of 62.5% of screened Schwarz's TM-APAP, 31.5% of the granule of Formulation E, and 1.0% of sodium stearyl fumarate.

These results indicated that the disintegration times are substantially improved comparing with the respective data shown in Example 8.

### Example 10

This example illustrates an alternative route of preparing fast disintegrating tablets using screened TM-APAP from Schwarz Pharma, namely making a premix containing granule Formulation A or B with a disintegrant of specified size.

92.5% of the granules of Formulation A was pre-blended with 7.5% (w/w) of calcium silicate (Zeopharm, HUBER engineered materials) in a 4 quart-V-blender for 20 minutes. (The blend was designated as G1). Similarly, 92.5% of the granule of Formulation B was pre-blended with 7.5% (w/w) of calcium silicate (Zeopharm, HUBER engineered materials) in a 4 quart-V-blender for 20 minutes (designated as G2). 1 Kg of final blends were prepared by mixing G1 or G2 respectively with 62.5% (w/w) of screened TM-APAP (equivalent to 50% pure APAP) with and without 5% of crospovidone XL10 in a 4 quart-V-blender for 20 minutes (see Table 10 for formulation compositions). The particle size of the screened TM-APAP was between 150 μm and 250 μm on sieve analysis (passed through a No. 60 sieve and retained on a No. 100 sieve). The final blends were then lubricated with 1.0% (w/w) of sodium stearyl fumarate (PRUV, JRS Pharma) for 5 minutes. The lubricated blends were compressed into tablets and the physical properties of the tablets were evaluated according to the procedures described in Example 2. The physical characteristics of the tablets are shown in Table 10.

TABLE 10

| | Hardness | Friability | USP-DIT | Wetting time |
|---|---|---|---|---|
| Tablet Formulation | (kP) | (%) | (sec.) * | (sec.) |
| Formulation 1 ^{a} | 9.1 | 0.14 | 29 | 22 |
| Formulation 2 ^{b} | 10.0 | 0.05 | 18 | 20 |
| Formulation 3 ^{c} | 9.1 | 0.23 | 24 | 19 |
| Formulation 4 ^{d} | 8.5 | 0.18 | 16 | 19 |

* In vitro disintegration time according to the USP method.
^{a} Formulation 1- tablets from a mixture of 62.5% (w/w) of screened Schwarz's TM-APAP, 36.5% (w/w) of G1, and 1.0% (w/w) of sodium stearyl fumarate.
^{b} Formulation 2- tablets from a mixture of 62.5% (w/w) of screened Schwarz's TM-APAP, 31.5% (w/w) of G1, 5% of crospovidone XL-10, and 1.0% (w/w) of sodium stearyl fumarate.
^{c} Formulation 3- tablets from a mixture of 62.5% (w/w) of screened Schwarz's TM-APAP, 36.5% (w/w) of G2, and 1.0% (w/w) of sodium stearyl fumarate.
^{d} Formulation 4- tablets from a mixture of 62.5% (w/w) of screened Schwarz's TM-APAP, 31.5% of G2, 5% (w/w) of crospovidone XL-10, and 1.0% (w/w) of sodium stearyl fumarate.

Comparing Formulation 1 using granule Formulation A in this Example with the high load formulation (62.5% TM-APAP) using the same granules in Example 5, clearly there was a significant improvement of disintegration time, 29 seconds for a 900mg tablet vs. 60 seconds for a 400 mg tablet respectively. Comparing Formulation 2 of this Example with the high load formulation (62.5% TM-APAP and 5% crosspovidone) in Example 5 the disintegration times were 18 seconds for a 900 mg tablet vs. 34 seconds for a 400 mg tablet respectively.

Comparing Formulation 3 using granule Formulation B and additional disintegrant of specified size in this Example with Formulation 1 in Example 8, clearly there was a significant improvement of disintegration time, 24 seconds vs. 47 seconds respectively. Comparing Formulation 4 using granule Formulation B and additional disintegrant of specified size in this Example with Formulation 1 in Example 9, clearly there was a further significant improvement of disintegration time, 16 seconds vs. 27 seconds respectively.

These results clearly indicated that 900 mg ODTs loaded with 62.5% TM-APAP (50% pure APAP) showed a disintegration time of less than 20 seconds according to the USP method, about 20 seconds in the Wetting test, and low friability of less 0.3% (w/w) with enough mechanical strength suitable for push through blister packages and regular HDPE bottles when the final ODT blend contained a certain amount of water insoluble inorganic salts with a particle size less than 50 μm and superdisintegrants such as regular calcium silicate and crospovidone XL-10. When less than 30% of APIs are added to the ODT blend, these two key components used in ODT with a high API load are optional. The preferred particle size range of coated APIs used for taste masking is between 100 μm and 250 μm for consistent compression during tableting and faster disintegration time.

What is claimed is:

1. An orally dissolving tablet comprising:
a) at least one water-insoluble hydrophobic inorganic salt in combination with at least one water-insoluble inorganic salt and
b) at least one active pharmaceutical ingredient,
wherein,
the orally dissolving tablet comprises up to about 70% w/w of active pharmaceutical ingredient in a unit dosage.

2. The tablet of claim 1 comprising:
a) 3% to 25% by weight of the at least one water-insoluble hydrophobic inorganic salt; and
b) 1% to 25% by weight of the at least one water-insoluble inorganic salt.

3. The tablet of claim 2 wherein the ratio of the at least one water-insoluble hydrophobic inorganic salt to the at least one water-insoluble inorganic salt is from about 1:10 to about 10:1.

4. The tablet of claim 1 further comprising:
a) at least one water-soluble excipient selected from the group consisting of sugar, sugar alcohols and mixtures thereof; and
b) at least one water-swellable polymeric material including a disintegrant;
wherein,
the at least one water-insoluble hydrophobic inorganic salt is selected from the group consisting of calcium diphosphate with a particle size of less than about 40 μm; calcium triphosphate: talc with a particle size less than 50 μm; and mixtures thereof; and
the at least one water-insoluble inorganic salt is selected from the group consisting of calcium silicate with a particle size of less than about 20 μm, hydrophobically modified calcium silicate with a particle size of less than 50 μm, talc with a particle size smaller than 80 μm and a mean particle size of 15 μm, and mixtures thereof.

5. The tablet of claim 4 wherein the disintegrant includes at least one modified starch selected from the group consisting of sodium starch glycolate, croscarmellose sodium, crospovidone, low substituted hydroxypropyl cellulose and mixtures thereof, and
the at least one water-soluble excipient is selected from the group consisting of sucrose, maltose, lactose, glucose, mannose, mannitol, sorbitol, xylitol, erythritol, lactitol, maltitol and mixtures thereof.

6. The tablet of claim 1 further comprising:
a) 18% to 88% by weight of at least one water-soluble excipient;
b) 1% to 20% by weight of at least one water insoluble/swellable polymeric material;
c) 3% to 25% by weight of the at least one water-insoluble hydrophobic inorganic salt; and
d) 1% to 25% by weight of the at least one water-insoluble inorganic salt.

7. The tablet of claim 1 wherein the at least one water-insoluble hydrophobic inorganic salt and the at least one water-insoluble inorganic salt have a particle size of no more than 80 μm.

8. The tablet of claim 1 further comprising at least one additive selected from the group consisting of colorants, sweeteners, flavorants, binders, lubricants and mixtures thereof.

9. The tablet of claim 1 wherein the at least one active pharmaceutical ingredient is selected from the group consisting of non-steroidal anti-inflammatory agents, contraceptives, opioids, thyroid and antithyroid drugs, gout therapy drugs, cough and cold drugs, anticonvulsants, antirheumatic drugs, anti-migraine drugs, anti-parasite, hormonal drugs, mitotic inhibitors, immunosuppressants, antihypersensitive agents, calcium-channel blocking agents, antidepressants, anxiolytics, neurodegenerative disease drugs, bismuth salts, coagulants, antiulcer agents, coronary vasodilators, peripheral vasodilators, oral antibacterial and antifungal agents, antispasmodics, antitussive agents, antiasthmatic agents, bronchodilators, diuretics, muscle relaxants, brain metabolism altering drugs, tranquilizers, beta blockers, antiarrhythmic agents, anticoagulants, antiepileptic agents, antiemetics, hypo- and hypertensive agents, sympathomimetic agents, expectorants, oral antidiabetic agents, circulatory agents, nutritional supplements, pollakiuria remedies, angiotension-converting enzyme inhibitors, antiviral agents, antihistamines, and nasal decongestants.

10. The tablet of claim 9 wherein the amount of at least one active pharmaceutical ingredient ranges from about 0.05% to about 70% w/w.

11. An orally dissolving tablet composition comprising:
a) 18% to 88% by weight of at least one water-soluble excipient;
b) 1% to 20% by weight of at least one water swellable polymeric material;
c) 3% to 25% by weight of at least one water-insoluble hydrophobic inorganic salt;
d) 1% to 25% by weight of at least one water-insoluble inorganic salt; and
e) at least one active pharmaceutical ingredient,
wherein the particle size of the water-insoluble hydrophobic inorganic salt and the water-insoluble inorganic salt is less than 80 μm; and
wherein the orally dissolving tablet comprises up to about 70% w/w of active pharmaceutical ingredient in a unit dosage.

12. The tablet of claim 11 wherein the ratio of the at least one water-insoluble hydrophobic inorganic salt to the at least one water-insoluble inorganic salt is from about 1:10 to about 10:1.

13. The tablet of claim 11 wherein:
a) the at least one water-soluble excipient is selected from the group consisting of sugar, sugar alcohols and mixtures thereof;
b) the at least one water-swellable polymeric material includes at least one disintegrant;
c) the at least one water-insoluble hydrophobic inorganic salt is selected from the group consisting of calcium diphosphate, calcium triphosphate, talc having a particle size less than 50 μm and mixtures thereof; and
d) the at least one water-insoluble inorganic salt is selected from the group consisting of physically modified calcium silicate and a talc having particle size smaller than 80 μm and a mean particle size of 15 μm and mixtures thereof.

14. The tablet of claim 11 wherein the disintegrant is selected from the group consisting of modified sodium starches, croscarmellose sodium, crospovidone, low substituted hydroxypropyl cellulose and mixtures thereof; and
the at least one water-soluble excipient is selected from the group consisting of sucrose, maltose, lactose, glucose, mannose, mannitol, sorbitol, xylitol, erythritol, lactitol, maltitol and mixtures thereof.

15. The tablet of claim 11 wherein the at least one active pharmaceutical ingredient is selected from the group consisting of non-steroidal anti-inflammatory agents, contraceptives, opioids, thyroid and antithyroid drugs, gout therapy drugs, cough and cold drugs, anticonvulsants, antirheumatic drugs, anti-migraine drugs, anti-parasite, hormonal drugs, mitotic inhibitors, immunosuppressants, antihypersensitive agents, calcium-channel blocking agents, antidepressants, anxiolytics, neurodegenerative disease drugs, bismuth salts, coagulants, antiulcer agents, coronary vasodilators, peripheral vasodilators, oral antibacterial and antifungal agents, antispasmodics, antitussive agents, antiasthmatic agents, bronchodilators, diuretics, muscle relaxants, brain metabolism altering drugs, tranquilizers, beta blockers, antiarrhythmic agents, anticoagulants, antiepileptic agents, antiemetics, hypo- and hypertensive agents, sympathomimetic agents, expectorants, oral antidiabetic agents, circulatory agents, nutritional supplements, pollakiuria remedies, angiotension-converting enzyme inhibitors, antiviral agents, antihistamines, and nasal decongestants.

16. The tablet of claim 15 wherein the amount of at least one active pharmaceutical ingredient ranges from about 0.05% to about 70% w/w.

17. An orally dissolving granule comprising:
a) 18% to 88% by weight of at least one water-soluble excipient;
b) 1% to 20% by weight of at least one water-swellable polymeric material;
c) 3% to 25% by weight of at least one water-insoluble hydrophobic inorganic salt; and
d) 1% to 25% by weight of at least one water-insoluble inorganic salt.

18. The granule of claim 17 wherein the at least one water-insoluble hydrophobic inorganic salt and the at least one water-insoluble inorganic salt have a particle size of less than about 80 μm.

19. The granule of claim 17 further comprising at least one active pharmaceutical ingredient.

20. The granule of claim 19 wherein the at least one active pharmaceutical ingredient is selected from the group consisting of non-steroidal anti-inflammatory agents, contraceptives, opioids, thyroid and antithyroid drugs, gout therapy drugs, cough and cord drugs, anticonvulsants, antirheumatic drugs, anti-migraine drugs, anti-parasite, hormonal drugs, mitotic inhibitors, immunosuppressants, antihypersensitive agents, calcium-channel blocking agents, antidepressants, anxiolytics, neurodegenerative disease drugs, bismuth salts, coagulants, antiulcer agents, coronary vasodilators, peripheral vasodilators, oral antibacterial and antifungal agents, antispasmodics, antitussive agents, antiasthmatic agents, bronchodilators, diuretics, muscle relaxants, brain metabolism altering drugs, tranquilizers, beta blockers, antiarrhythmic agents, anticoagulants, antiepileptic agents, antiemetics, hype and hypertensive agents, sympathomimetic agents, expectorants, oral antidiabetic agents, circulatory agents, nutritional supplements, pollakiuria remedies, angiotension-converting enzyme inhibitors, antiviral agents, antihistamines, and nasal decongestants.

21. The granule of claim 20 wherein the amount of at least one active pharmaceutical ingredient ranges from about 0.05% to about 70% w/w.

22. The granule of claim 17 wherein:
a) the at least one water-soluble excipient is selected from the group consisting of sugar, sugar alcohols and mixtures thereof;
b) the at least one water-swellable polymeric material includes at least one disintegrant;
c) the at least one water-insoluble hydrophobic inorganic salt is selected from the group consisting of calcium diphosphate, calcium triphosphate, talc with a particle size less than about 50 μm, and mixtures thereof; and
d) the at least one water-insoluble inorganic salt is selected from the group consisting of modified calcium silicate and talc with a particle size smaller than about 80 μm and a mean particle size of about 15 μm and mixtures thereof.

23. The granule of claim 22 wherein the at least one selected disintegrant is selected from the group consisting of modified starch, croscarmellose sodium, crospovidone, low substituted hydroxypropyl cellulose and mixtures thereof; and the water-soluble excipient is selected from the group consisting of sucrose, maltose, lactose, glucose, mannose, mannitol, sorbitol, xylitol, erythritol, lactitol, maltitol, and mixtures thereof.

24. The granule of claim 17 further comprising at least one additive selected from the group consisting of colorants, sweeteners, flavorants, binders, lubricants, and mixtures thereof.

25. A method of making orally dissolving granules, the method comprising
a) granulating a mixture including:
i) at least one water-soluble excipient;
ii) at least one water swellable polymeric material;
iii) at least one water-insoluble hydrophobic inorganic salt; and
iv) at least one water-insoluble inorganic salt;
with water or a polymeric binder solution to form wet granules;
b) drying the wet granules to form substantially dry granules; and
c) screening (or milling) the substantially dry granules to produce orally dissolving granules of a desired size.

26. The method of claim 25 wherein the polymeric binder solution comprises fully pregelatinized starch and water soluble polymers.

27. The method of claim 25 comprising from about 0.5% to about 15% by weight of at least one water soluble polymeric binder.

28. The method of claim 25 wherein the particle size of the orally dissolving granules is less than about 700 μm.

29. The method of claim 25 wherein the orally disintegrating granules further comprise at least one active pharmaceutical ingredient.

30. The method of claim 29 wherein the orally disintegrating granules comprise at least 30% by weight of at least one active pharmaceutical ingredient.

31. The method of claim 30 wherein the orally disintegrating granules have a bimodal particle size distribution consisting of a first mode representing a particle size group of 1 μm-80 μm and a, second mode representing a particle size group of 70 μm-700 μm.

32. The method of claim 30 further comprising adding at least one water swellable polymeric material and at least one water-insoluble inorganic salt to the substantially dried granules having an unimodal particle size distribution pattern in the final blend before lubrication.

33. The method of claim 32 comprising:
a) 1% to 10% by weight of at least one water-insoluble inorganic salt with a particle size of 0.5 μm-20 μm; and
b) 1% to 15% by weight of at least one water swellable polymeric material with a particle size of 0.5 μm-80 μm.

34. The method of claim 32 wherein:
a) the at least one water-insoluble inorganic salt is selected from the group consisting of regular calcium silicate, talc, and dicalcium phosphate and mixtures thereof; and
b) the at least one water swellable polymeric material comprises at least one disintegrant.

35. The method of claim 25 further comprising adding a lubricant to the substantially dried granules.

36. The method of claim 25 further comprising adding at least one active pharmaceutical ingredient to the substantially dried granules.

37. The method of claim 25 wherein the mixture includes:
a) 18% to 88% by weight of the at least one water-soluble excipient;
b) 1% to 20% by weight of the at least one water-swellable-polymeric material;
c) 3% to 25% by weight of the at least one water-insoluble hydrophobic inorganic salt; and
d) 1% to 25% by weight of the at least one water-insoluble inorganic salt.

38. A method of making orally dissolving granules, the method comprising
a) granulating a mixture including:
i) 18% to 90% by weight of at least one water-soluble excipient;
ii) 1% to 20% by weight of at least one water swellable polymeric material;
iii) 3% to 25% by weight of at least one water-insoluble hydrophobic inorganic salt; and
iv) ¹% to 25% by weight at least one water-insoluble inorganic salt with water to form wet granules;
b) drying the wet granules to form substantially dry granules; and
c) milling the substantially dry granules to produce orally dissolving granules of a desired size.

39. The method of claim 38 wherein the ratio of the at least one water-insoluble hydrophobic inorganic salt to the at least one water-insoluble inorganic salt is from about 1:10 to about 10:1.

40. The method of claim 38 wherein the particle size of the orally dissolving granules is no more than about 700 μm.

41. A method of making a orally dissolving tablet, the method comprising:
a) granulating a mixture including
i) 18% to 88% by weight of at least one water-soluble excipient;
ii) 1% to 20% by weight of at least one water swellable polymeric material;
iii) 3% to 25% by weight of at least one water-insoluble hydrophobic inorganic salt; and
iv) 1% to 25% by weight of at least one water-insoluble inorganic salt with water to form wet granules;
b) drying the wet granules to form substantially dry granules;
c) screening (or milling) the substantially dry granules to produce orally dissolving granules of a desired size; and
d) compressing the granules to form a tablet.

42. The method of claim 41 wherein the ratio of the at least one water-insoluble hydrophobic inorganic salt to the at least one water-insoluble inorganic salt is from about 1:10 to about 10:1.

43. The method of claim 41 wherein the particle size of the at least one water-insoluble hydrophobic inorganic salt and the at least one water-insoluble inorganic salt is no more then about 80 μm.

44. The method of claim 41 wherein the mean particle size of the substantially dry granules is from about 100 μm to about 200 μm.

45. The method of claim 41 further comprising adding a lubricant to the granules prior to compressing the granules into a tablet.

46. The method of claim 41 further comprising adding at least one active pharmaceutical ingredient.

47. The method of claim 46 wherein the at least one active pharmaceutical ingredient is selected from the group consisting of non-steroidal anti-inflammatory agents, contraceptives, opioids, thyroid and antithyroid drugs, gout therapy drugs, cough and cold drugs, anticonvulsants, antirheumatic drugs, anti-migraine drugs, anti-parasite, hormonal drugs, mitotic inhibitors, immunosuppressants, antihypersensitive agents, calcium-channel blocking agents, antidepressants, anxiolytics, neurodegenerative disease drugs, bismuth salts, coagulants, antiulcer agents, coronary vasodilators, peripheral vasodilators, oral antibacterial and antifungal agents, antispasmodics, antitussive agents, antiasthmatic agents, bronchodilators, diuretics, muscle relaxants, brain metabolism altering drugs, tranquilizers, beta blockers, antiarrhythmic agents, anticoagulants, antiepileptic agents, antiemetics, hypo and hypertensive agents, sympathomimetic agents, expectorants, oral antidiabetic agents, circulatory agents, nutritional supplements, pollakiuria remedies, angiotension-converting enzyme inhibitors, antiviral agents, antihistamines, and nasal decongestants.

48. The method of claim 46 wherein the amount of at least one active pharmaceutical ingredient ranges from about 0.05% to about 70% w/w.
